(19) United States
(12) Patent Application Publication
(10) Pub. No.: US 2026/0263767 A1
(43) Pub. Date: Sep. 10, 2026

(54) MICRONEEDLE PATCH

(71) Applicant: DARWIN PRECISIONS CORPORATION, Hsinchu County (TW)

(72) Inventors: Yi-Chen Liu, Hsinchu County (TW); WEI-TANG CHENG, Hsinchu County (TW); Yun-Pei Yang, Hsinchu County (TW)

(21) Appl. No.: 19/557,171

(22) Filed: Mar. 5, 2026

(30) Foreign Application Priority Data
Mar. 7, 2025 (TW) 114108594

(51) Int. Cl. A61M 37/00

(52) U.S. Cl. CPC A61M 37/0015; A61M 2037/0023; A61M 2037/0046; A61M 2037/0053

(57) ABSTRACT

A microneedle patch including a microneedle film and a carrier layer is provided. The microneedle film includes a base layer and a plurality of microneedles, and the microneedle film is formed of at least a film-forming material and a water-soluble material. The plurality of microneedles are spaced apart from each other on the base layer, a molecular weight of the film-forming material is greater than 100,000 Da, a molecular weight of the water-soluble material is not greater than 100,000 Da, and a weight ratio of the film-forming material to the water-soluble material is from 2:1 to 4:5. The base layer has a first surface and a second surface opposite to each other, and the plurality of microneedles are located on the first surface, and the microneedle film is disposed on the carrier layer via the second surface of the base layer.

### FIELD OF THE INVENTION

The present invention relates to the field of transdermal drug delivery, and more particularly to a microneedle patch.

### BACKGROUND OF THE INVENTION

Microneedle patches (MNPs) are a novel type of transdermal drug delivery system (TDDS). The microneedles on the patch are extremely short and do not penetrate deep enough to contact nerve endings, thereby avoiding the pain associated with subcutaneous injections. Also, MNPs are capable of delivering bioactive agents or pharmaceutical compounds through the stratum corneum of the skin into the human body. The MNP technology may be applied in various fields, including aesthetic medicine, pharmaceuticals, and preventive healthcare.

### SUMMARY OF THE INVENTION

The present invention provides a microneedle patch that helps reduce defects during the manufacturing process, thereby improving yield and production efficiency.

In order to achieve one, part, or all of the above objects or other objects, the present invention provides a microneedle patch including a microneedle film and a carrier layer. The microneedle film includes a base layer and a plurality of microneedles. The microneedle film includes at least a film-forming material and a water-soluble material. The microneedles are disposed on the base layer and spaced apart from each other. The molecular weight of the film-forming material is greater than 100,000 Da. The molecular weight of the water-soluble material is not greater than 100,000 Da. The weight ratio of the film-forming material to the water-soluble material is from 2:1 to 4:5. The base layer includes a first surface and a second surface opposite to the first surface. The microneedles are disposed on the first surface. The microneedle film is disposed on the carrier layer, with the second surface of the base layer facing the carrier layer.

In one embodiment of the present invention, the microneedle film further includes a hardening material. The molecular weight of the hardening material is less than 100,000 Da.

By using the film-forming material and the water-soluble material to form the microneedle film, the present invention can improve the filling and deaerating ability during the fabrication process. As such, it effectively reduces the occurrence of empty needles, residual bubbles, and voids, thereby forming microneedles with structural integrity and excellent skin penetration performance. Accordingly, the invention enhances the yield and production efficiency of the microneedle patches.

Other objectives, features and advantages of the invention will be further understood from the further technological features disclosed by the embodiments of the invention wherein there are shown and described preferred embodiments of this invention, simply by way of illustration of modes best suited to carry out the invention.

### BRIEF DESCRIPTION OF THE DRAWINGS

FIG. 1 is a schematic cross-sectional view of a microneedle patch according to an embodiment of the invention; and

FIG. 2 is a schematic diagram showing microneedle dissolution rates over time of the microneedles in the embodiment and in the comparison sample.

### DETAILED DESCRIPTION OF PREFERRED EMBODIMENTS

In the following description of embodiments according to the present invention, terms indicating orientation or positional relationships, such as “upper,” “lower,” etc., are described based on the orientation or positional relationships shown in the corresponding drawings. These terms are used solely for the convenience of describing the invention and are not intended to limit the invention or to imply that the referenced elements must have a particular orientation or be constructed in a particular orientation. Furthermore, the terms “first,” “second,” and the like, as used in this specification or the claims, are employed merely to designate names of elements or to distinguish between different embodiments or ranges and are not intended to limit the number of elements. Unless limited otherwise, the terms “connected” or “coupled” as used in this specification or the claims may refer to either a direct connection between two elements without the presence of other elements or an indirect connection through the presence of other elements.

The present invention provides a microneedle patch cable of reducing defects during the manufacturing process, thereby improving yield and production efficiency. The microneedle patch of the present invention delivers active ingredients through microneedle penetration and may be used for transdermal drug delivery or transdermal absorption as a carrier for various active ingredients. The active ingredients may include bioactive ingredients, pharmaceutical ingredients, or a combination thereof. In the present invention, the term “bioactive ingredients” refers to a substance capable of producing a biological effect in humans or other organisms, such as altering physiological functions or biochemical metabolism. The bioactive ingredients are not limited to those achieving therapeutic or preventive effects. However, bioactive ingredients with therapeutic or preventive effectiveness may be regarded as pharmaceutical ingredients.

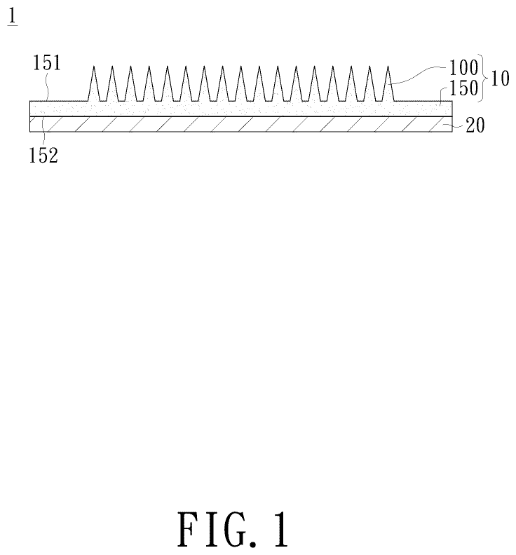

FIG. 1 is a schematic cross-sectional view of a microneedle patch according to an embodiment of the invention. As shown in FIG. 1, a microneedle patch 1 includes a microneedle film 10 and a carrier layer 20. The microneedle film 10 includes a base layer 150 and a plurality of microneedles 100. The microneedles are disposed on the same side of the base layer 150 and spaced apart from each other. A diameter of the microneedle 100 increases gradually toward the base layer 150. Specifically, the microneedle 100 may have a shape of a pyramid or a cone. A length of the microneedle 100 is between 100 μm and 1500 μm, such as 100 μm, 500 μm, 800 μm, 1000 μm, and 1500 μm. The base layer 150 has a thickness between 5 μm and 50 μm and includes a first surface 151 and a second surface 152. The microneedles 100 are disposed on the same surface, such as the first surface 151. The other surface, such as the second surface 152, may be utilized to connect the microneedle film 10 and the carrier layer 20. The carrier layer 20 may be adapted to carry, support, or protect the microneedle film 10. The material of the carrier layer 20 may be, for example, a natural or synthetic fabric such as cloth, or a natural or synthetic polymer such as a film. The carrier layer 20 may further serve to secure the microneedle film 10 onto the epidermis during the transdermal delivery. Accordingly, the material of the carrier layer 20 may also be selected based on various functional considerations, such as water-resistance, breathability, antimicrobial properties, odor control, aesthetics, or tactile feel.

In an embodiment of the present invention, the microneedle film 10 includes at least a film-forming material and a water-soluble material. The film-forming material is adapted for formation of the microneedle film 10 and possesses biocompatibility. Biocompatibility means the material does not release toxic or harmful substances that may cause local or systemic adverse effects in the human body or other organisms, such as inflammatory reactions, immune responses, or toxicological effects. For example, the film-forming material includes, but is not limited to, hyaluronic acid, polyvinylpyrrolidone (PVP), polyvinyl alcohol (PVA), polylactic acid (PLA), chitosan, carboxymethyl cellulose (CMC), and chondroitin sulfate. The film-forming material further exhibits degradability (or solubility). When the microneedle film 10 and the microneedles 100 formed from the film-forming material are used for transdermal delivery, the microneedles 100 degrade (also referred to as dissolve) over time without causing adverse effects to the human body or other organisms.

In the present embodiment of the present invention, the film-forming material has a relatively high molecular weight. A macromolecular material may be used as the film-forming material. The film-forming material may have a molecular weight greater than 100,000 Da and may form a polymer. As used herein, “molecular weight” refers to the relative molecular weight and may further refer to the weight-average molecular weight (Mw). The weight-average molecular weight of the film-forming material may be between 100,000 Da and 20,000,000 Da, such as 150,000 Da, 200,000 Da, 300,000 Da, 400,000 Da, 500,000 Da, 600,000 Da, 700,000 Da, 800,000 Da, 900,000 Da, 1,000,000 Da, 2,000,000 Da, 3,000,000 Da, 4,000,000 Da, 5,000,000 Da, 6,000,000 Da, 7,000,000 Da, 8,000,000 Da, 9,000,000 Da, 10,000,000 Da, 15,000,000 Da, and 20,000,000 Da. A mixed solution containing the film-forming material and the water-soluble material can be made. For example, a film-forming solution including the film-forming material and the water-soluble material may be prepared by using an aqueous solvent. The aqueous solvent may be, for example, water. The film-forming solution may be formed into the microneedle film 10 through steps including molding (injection into a mold), drying, and so on.

The water-soluble material of the microneedle film 10 has a molecular weight less than the molecular weight of the film-forming material. The water-soluble material may be a material characterized as a bioactive ingredient, a pharmaceutical ingredient, or a combination thereof. In other words, the water-soluble material may further include the aforementioned bioactive ingredient, pharmaceutical ingredient, or a combination thereof, and thus serves as the active ingredient in the present invention. The water-soluble material serving as the active ingredient may be carried by the microneedle film 10 and the microneedles 100, and may be transdermally delivered into the human body or other organisms. Furthermore, the water-soluble material may be released in vivo as the microneedles 100 degrade, thereby exerting the intended effect.

In the embodiment of the present invention, the molecular weight of the water-soluble material is less than or equal to 100,000 Da. In one embodiment, the molecular weight of the water-soluble material is between 500 Da and 100,000 Da, such as 500 Da, 1,000 Da, 2,000 Da, 3,000 Da, 4,000 Da, 5,000 Da, 6,000 Da, 7,000 Da, 8,000 Da, 9,000 Da, 10,000 Da, 20,000 Da, 30,000 Da, 40,000 Da, 50,000 Da, 60,000 Da, 70,000 Da, 80,000 Da, 90,000 Da, and 100,000 Da. In another embodiment, the molecular weight of the water-soluble material is between 500 Da and 10,000 Da, such as 500 Da, 1,000 Da, 1,500 Da, 2,000 Da, 2,500 Da, 3,000 Da, 3,500 Da, 4,000 Da, 4,500 Da, 5,000 Da, 5,500 Da, 6,000 Da, 6,500 Da, 7,000 Da, 7,500 Da, 8,000 Da, 8,500 Da, 9,000 Da, 9,500 Da, and 10,000 Da.

Specifically, the water-soluble material may include an organic compound, an inorganic compound, an extract, or a combination thereof. The aforementioned organic compound, inorganic compound, and extract (e.g., an extract from organisms) may be a natural or synthetic compound and preferably include a bioactive ingredient, a pharmaceutical ingredient, or a combination thereof. For example, the water-soluble material may include, but is not limited to, proteins, peptides, amino acids, other organic acids or derivatives thereof, polysaccharides or derivatives thereof, vitamins, alcohol-based compounds, amine compounds, amide compounds, pharmaceutical ingredients, or a combination thereof. Also, the water-soluble material may include more than one material. In other words, multiple water-soluble materials may be utilized together in the microneedle film 10. In the embodiment of the present invention, the water-soluble material may be, for example, proteins such as collagen, peptides such as collagen peptides, and other compounds having functional effectiveness such as anti-wrinkle, anti-aging, moisturizing properties, or combinations thereof.

In addition to serving as an active ingredient, the water-soluble material may also cooperate with the film-forming material to optimize the manufacturing process and improve the yield of the microneedle film 10 formation. Specifically, when the microneedle film 10 is formed with the film-forming material and the water-soluble material, a proper proportion of the water-soluble material may help mitigate the high viscosity issue of the film-forming solution caused by the film-forming material. Furthermore, during the formation of the microneedle 10, the film-forming solution is typically treated with steps of molding (i.e., filling the solution into a mold), deaeration, and so on. Thus, the viscosity of the film-forming solution may significantly affect the difficulty of the mold filling and the effectiveness of the deaeration, leading to an impact on the formation of the microneedle film 10 and the overall production yield. Since the water-soluble material may lower the viscosity of the film-forming solution, a proper proportion of the water-soluble material may lower the viscosity of the film-forming solution to a sufficient extent, thereby facilitating the mold filling, improving the deaeration, and enabling the effective removal of bubbles in the solution. On the contrary, if the amount of the water-soluble material is less than an amount required for the proper proportion, issues such as incomplete mold filling, ineffective deaeration, and insufficient bubble removal may arise. Such deficiencies can result in an incomplete structure of the microneedle film 10, defective formation of the microneedle film 10, or formation failure of the microneedle film 10, including the presence of empty microneedles, residual bubbles, or structural voids.

It is noted that if the proportion of the water-soluble material exceeds the aforementioned proper proportion and the proportion of the film-forming material becomes relatively insufficient, it may lead to a decrease in the mechanic strength of the microneedle film 10. For example, it further results in defects such as brittleness and breakage of the microneedles 100 or cracking of the base layer 150. The appropriate proportion of the water-soluble material may be expressed in terms of weight percentage or weight ratio relative to the film-forming material. In the embodiment of the present invention, a weight ratio of the film-forming material to the water-soluble material is from 2:1 to 4:5, such as 2:1, 6:5, 5:4, 1:1, 8:9, and 4:5.

In addition, the film-forming solution may further include other active ingredients, such that the formed microneedle film 10 may provide additional or enhanced functions. The active ingredients may include proteins, peptides, amino acids, other organic acids or derivatives thereof, polysaccharides or derivatives thereof, vitamins, alcohol-based compounds, amine-based compounds, amide-based compounds, pharmaceutical agents, or a combination thereof. The invention is not limited thereto. The active ingredients may include ergothioneine (EGT), tranexamic acid, salicylic acid, ceramides, niacinamide, vitamin B complex, vitamin C, Ganoderma lucidum polysaccharides, Astragalus polysaccharides, Polygonatum odoratum polysaccharides, Rehmannia glutinosa polysaccharides, and various medicines. The functional effects of such ingredients may include, but are not limited to, skin whitening, anti-aging, anti-acne, antioxidation, anti-inflammation, maintenance or enhancement of immune system function, analgesia, sedation, antipyresis, or a combination thereof.

In another embodiment of the present invention, in addition to the film-forming material and the water-soluble material, the microneedle film 10 further includes a hardening material. The hardening material is adapted to modify the film-forming properties and preferably, to enhance the ductility, flexibility, and elasticity of the microneedle film 10. For example, the hardening material may be intercalated between the polymer molecular chains formed by the film-forming material, thereby reducing the intermolecular forces among the polymer molecular chains. It further increases the free volume and mobility of the molecular chains, thereby enhancing the ductility, flexibility, and elasticity of the microneedle film 10.

The hardening materials may have a relatively low molecular weight. In the embodiment of the present invention, the molecular weight of the hardening material is, for example, less than or equal to 100,000 Da. The molecular weight of the hardening material is less than the molecular weight of the water-soluble material. The invention is not limited thereto. Specifically, the hardening material may include monosaccharides, disaccharides, oligosaccharides, polyols, lipids or derivatives thereof, or a combination thereof. The hardening material is not limited to a single material. In other words, the hardening material may include multiple materials for forming the microneedle film 10. The molecular weight of the hardening material may be, for example, less than 500 Da, although it is not limited thereto. In an embodiment of the present invention, the hardening material may include, for example, fructose, glucose, trehalose, honey, glycerol, sorbitol, xylitol, maltitol, diglycerol, or dipropylene glycol.

Plasticizers, humectants, and certain polymeric compounds, such as polyethylene glycol and polyglutamic acid, may also serve as the hardening material. In some embodiments, the polyglutamic acid used may have either a relatively high molecular weight, such as tens of millions, or a relatively low molecular weight, such as several millions. For example, the molecular weight of the polyglutamic acid may range from 10,000,000 Da to 12,000,000 Da, or in a range from 2,000,000 Da to 4,000,000 Da.

In addition to modifying the film-forming properties, the amount of the water-soluble material may be increased accordingly due to the usage of the hardening material. As such, the microneedle film 10 may exhibit improved extensibility, flexibility, and elasticity, thereby mitigating the inherent brittleness and the tendency to fracture. In addition, the amount of active ingredients contained in the microneedle film 10 may be further increased, thereby facilitating the mold filling and enhancing the efficiency of bubble removal. The aforementioned weight ratio of the film-forming material to the water-soluble material, preferably between 2:1 and 4:5, is appropriate under the condition that the hardening material is also included in the microneedle film 10. In the embodiment of the present invention, the weight ratio of the sum of the film-forming material and the water-soluble material to the hardening material is between 9.5:0.5 and 8:2, such as 9.5:0.5, 9:1, 8.8:1.2, 8.5:1.5, 8.2:1.8, and 8:2. In addition, the hardening material may have the properties shown in the water-soluble material. Under this condition, the hardening material may be regarded as the water-soluble material and used as such. The weight ratio of the film-forming material to the hardening material (the water-soluble material) is preferably between 2:1 and 4:5. The weight ratio of the sum of the film-forming material and the hardening material (the water-soluble material) to the hardening material is preferably between 9.5:0.5 and 8:2.

Please refer to Example 1 and Example 2, which show specific examples of the film-forming material, the water-soluble material, and the hardening material.

### Example 1

In Example 1, the hyaluronic acid having a molecular weight of 200,000 Da is used as the film-forming material, the trehalose (having a molecular weight of less than 500 Da) is used as the hardening material, and the hydrolyzed collagen having a molecular weight of 3,000 Da is used as the water-soluble material. The weight ratios among the hyaluronic acid, the trehalose, and the hydrolyzed collagen are shown in Table 1. In Examples 1-1 to 1-5, the hardening material is not used. In Examples 1-1 to 1-8, the film-forming solutions are prepared individually, followed by steps of molding (filling), deaeration, and drying to form the microneedle films 10.

TABLE 1

| | Hyaluronic | Hydrolyzed | | | | |
|---|---|---|---|---|---|---|
| | acid | collagen | Trehalose | | | |
| | (weight | (weight | (weight | Poor | Brittle | Re- |
| | parts) | parts) | parts) | deaeration | crack | sults |
| 1-1 | 9 | 1 | 0 | Yes | No | NG |
| 1-2 | 8 | 2 | 0 | Yes | No | NG |
| 1-3 | 7 | 3 | 0 | Yes | Yes | NG |
| 1-4 | 6 | 4 | 0 | No | Yes | NG |
| 1-5 | 5 | 5 | 0 | No | Yes | NG |
| 1-6 | 6 | 3 | 1 | No | No | OK |
| 1-7 | 4.8 | 4 | 1.2 | No | No | OK |
| 1-8 | 4 | 4.5 | 1.5 | No | No | OK |

As shown from Examples 1-1 to 1-5, as the amount of the hydrolyzed collagen increases, the issue of poor deaeration is overcome while the issue of brittle crack starts to occur accordingly. As shown in Example 1-4, when the weight ratio of the hyaluronic acid to the hydrolyzed collagen becomes 6:4, the issue of poor deaeration is solved, but the issue of brittle crack occurs. As shown in Examples 1-6 to 1-8, after the trehalose is added, the issues of poor deaeration and brittle crack are both solved. When the weight ratio of the hyaluronic acid to the hydrolyzed collagen becomes 4.8:4 (Example 1-7), which is close to 1:1, or when the amount of the hydrolyzed collagen is further increased to exceed the amount of the hyaluronic acid (Example 1-8), no brittle crack is observed on the microneedle film 10 surface. In contrast, as shown in Example 1-5, in the absence of the trehalose, when the amount of the hydrolyzed collagen is increased to be comparable to the amount of the hyaluronic acid (5:5), poor formation of the microneedle film 10 is observed.

### Example 2

In Example 2, the hyaluronic acid having a molecular weight of 850,000 Da is used as the film-forming material, the polyglutamic acid is used as the hardening material, and the collagen peptides having a molecular weight of 500 Da is used as the water-soluble material. The weight ratios among the hyaluronic acid, the collagen peptides, and the polyglutamic acid are shown in Table 2. In Examples 2-1 to 2-5, the hardening material is not used. In Examples 2-1 to 2-8, the film-forming solutions are prepared individually, followed by steps of molding (filling), deaeration, and drying to form the microneedle films 10.

TABLE 2

| | Hyaluronic | Collagen | polyglutamic | | | |
|---|---|---|---|---|---|---|
| | acid | peptides | acid | | | |
| | (weight | (weight | (weight | Poor | Brittle | Re- |
| | parts) | parts) | parts) | deaeration | crack | sults |
| 2-1 | 9 | 1 | 0 | Yes | No | NG |
| 2-2 | 8 | 2 | 0 | Yes | No | NG |
| 2-3 | 7 | 3 | 0 | Yes | Yes | NG |
| 2-4 | 6 | 4 | 0 | No | Yes | NG |
| 2-5 | 5 | 5 | 0 | No | Yes | NG |
| 2-6 | 6 | 3 | 1 | No | No | OK |
| 2-7 | 5 | 4 | 1 | No | No | OK |
| 2-8 | 4 | 5 | 1 | No | No | OK |

As shown in Examples 2-1 to 2-5, as the amount of the collagen peptides increases, the issue of poor deaeration is overcome while the issue of brittle crack starts to occur accordingly. When the weight ratio of the hyaluronic acid to the collagen peptides becomes 6:4, the issue of poor deaeration is solved, but the issue of brittle crack occurs. As shown in Examples 2-6 to 2-8, after the polyglutamic acid is added, the issues of poor deaeration and brittle crack are both solved. When the weight ratio of the hyaluronic acid to the collagen peptides becomes 5:4 (Example 2-7), which is close to 1:1, or when the amount of the collagen peptides is further increased to exceed the amount of the hyaluronic acid (Example 2-8), no brittle crack is observed on the microneedle film 10 surface. In contrast, as shown in Example 2-5, in the absence of the polyglutamic acid, when the amount of the collagen peptides is increased to be comparable to the amount of the hyaluronic acid (5:5), poor formation of the microneedle film 10 is observed.

Accordingly, the microneedle film 10 in the embodiment of the present invention is well formed with sufficient ductility, flexibility, and elasticity, without issues such as brittleness or fracture. The amount of the water-soluble material contained in the microneedle film 10 can also be increased. As a result, the microneedles 100 formed in this embodiment can effectively penetrate the skin with high delivery efficiency, thereby enhancing the performance of the active ingredients. Furthermore, the microneedle film 10 of the present invention can be used to accelerate the release of active ingredients, achieving the desired effects within a shorter period of time.

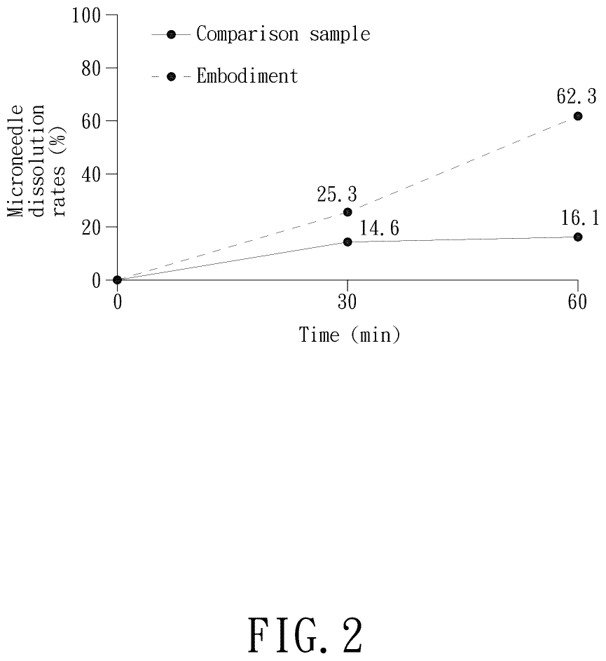

FIG. 2 is a schematic diagram showing the microneedle dissolution rates over time of the microneedles in the embodiment and in the comparison sample. The microneedle patch 1 in FIG. 2 includes the microneedle film 10 formed of the film-forming material, the water-soluble material, and the hardening material. The weight ratio of the film-forming material to the water-soluble material is between 2:1 and 4:5, and the weight ratio of the sum of the film-forming material and the water-soluble material to the hardening material is between 9.5:0.5 and 8:2. The microneedle film in the comparison sample is formed of the film-forming material only. The microneedle dissolution rate is defined as a weight percentage of the dissolved microneedles to the total microneedles on the microneedle film. As shown in FIG. 2, in the present embodiment, due to the relatively lower amount of the film-forming material, the degradation can proceed at an accelerated rate. For example, the dissolution rate of the microneedle 100 within 1 hour (60 minutes) may be greater than 50%, such as 62.3%. In contrast, the dissolution rate of the comparison sample within 1 hour is less than 20%. In addition, in the embodiment of the present invention, the microneedle dissolution rate exhibits an approximately linear correlation with time, indicating that the microneedle dissolution rate increases steadily over time. In the comparison sample, the microneedle dissolution rate after one hour increases by only 2% compared to the microneedle dissolution rate at 30 minutes. Based on this, the microneedle patch 1 of the present invention can also be applied to active ingredients which require rapid release, providing a steady release effect within a specific period of time. On the other hand, the amount of the water-soluble material is further increased, thereby enabling the microneedle patch 1 of the present invention to deliver a significant effect of active ingredients within a shorter period of time.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A microneedle patch, comprising:
a microneedle film; and
a carrier layer;
wherein the microneedle film comprises a base layer and a plurality of microneedles, the microneedle film comprises at least a film-forming material and a water-soluble material, the microneedles are spaced apart from each other and disposed on the base layer, a molecular weight of the film-forming material is greater than 100,000 Da, a molecular weight of the water-soluble material is not greater than 100,000 Da, a weight ratio of the film-forming material to the water-soluble material is from 2:1 to 4:5,
the base layer comprises a first surface and a second surface opposite to each other, the microneedles are disposed on the first surface, and the microneedle film is disposed on the carrier layer, with the second surface of the base layer facing the carrier layer.

2. The microneedle patch according to claim 1, wherein the molecular weight of the film-forming material is defined as a weight-average molecular weight, and the molecular weight of the film-forming material is between 100,000 Da and 20,000,000 Da.

3. The microneedle patch according to claim 1, wherein the molecular weight of the water-soluble material is between 500 Da and 100,000 Da.

4. The microneedle patch according to claim 3, wherein the molecular weight of the water-soluble material is between 500 Da and 10,000 Da.

5. The microneedle patch according to claim 3, wherein the microneedle film further comprises a hardening material.

6. The microneedle patch according to claim 5, wherein a molecular weight of the hardening material is less than the molecular weight of the water-soluble material.

7. The microneedle patch according to claim 5, wherein a weight ratio of the sum of the film-forming material and the water-soluble material to the hardening material is from 9.5:0.5 to 8:2.

8. The microneedle patch according to claim 5, wherein the hardening material comprises a monosaccharide, a disaccharide, an oligosaccharide, a polyol, a lipid or a derivative thereof, a plasticizer, a humectant, or a combination thereof.

9. The microneedle patch according to claim 8, wherein the hardening material further comprises a polyglutamic acid.

10. The microneedle patch according to claim 1, wherein the water-soluble material is a bioactive ingredient.

11. The microneedle patch according to claim 10, wherein the water-soluble material comprises a natural or synthetic organic compound, a natural or synthetic inorganic compound, an extract, or a combination thereof.

12. The microneedle patch according to claim 1, wherein the microneedle film further comprises an active ingredient.

13. The microneedle patch according to claim 1, wherein the microneedles are dissolvable, and a microneedle dissolution rate within one hour is greater than 50%, where the microneedle dissolution rate is defined as a weight percentage of the dissolved microneedles relative to the total microneedles in the microneedle film.
